(19) United States
(12) Patent Application Publication
(10) Pub. No.: US 2017/0035880 A1
(43) Pub. Date: Feb. 9, 2017

(54) LIPOTEICHOIC ACID FROM LACTOBACILLI AS A POTENT IMMUNE STIMULATORY ADJUVANT FOR VACCINE DEVELOPMENT

(71) Applicant: UNIVERSITY OF FLORIDA RESEARCH FOUNDATION, INCORPORATED, GAINESVILLE, FL (US)

(72) Inventors: MANSOUR MOHAMADZADEH, GAINESVILLE, FL (US); BIKASH SAHAY, GAINESVILLE, FL (US); SHAHRAM SALEK-ARDAKANI, NEWBERRY, FL (US); VIKAS TAHILIANI, GAINESVILLE, FL (US)

(21) Appl. No.: 15/303,760

(22) PCT Filed: Apr. 23, 2015
(86) PCT No.: PCT/US15/27221

§ 371 Date: Oct. 13, 2016

Related U.S. Application Data
(60) Provisional application No. 61/983,156, filed on Apr. 23, 2014.

(51) Int. Cl. A61K 39/39; A61K 39/395; C07K 16/28

(52) U.S. Cl. CPC A61K 39/39; C07K 16/2875; A61K 39/3955; A61K 2039/55572

(57) ABSTRACT

The present invention provides compositions and methods useful for vaccination and generating CD8+ T lymphocyte immune memory against one or more antigens utilizing lipoteichoic acid.

### CROSS-REFERENCE TO RELATED APPLICATION

This application claims the benefit of U.S. Provisional Application Ser. No. 61/983,156, filed Apr. 23, 2014, the disclosure of which is hereby incorporated by reference in its entirety, including all figures, tables and amino acid or nucleic acid sequences.

### BACKGROUND OF THE INVENTION

To expedite the process of vaccine development, pure recombinant or synthetic antigens are used in modern vaccines; however, in general, they are far less immunogenic than conventional vaccines containing live or killed whole organisms. This has created a major need for improved and more powerful adjuvants for use in these vaccines. With few exceptions, alum remains the sole adjuvant approved for human use in the majority of countries worldwide. Although alum is able to induce a good antibody (Th2) response, it has little capacity to stimulate cellular (Th1) immune responses, which are vital for protection against many pathogens. Most pathogens enter the body via mucosal surfaces. Current ideas support the notion that more centralized memory T cells, which circulate throughout secondary lymphoid organs, will not respond, expand in number, or relocate quickly enough to provide immediate protection against diseases caused by pathogen reinfection. In contrast, memory T cells that populate peripheral organs, such as the lung and gut, sometimes referred to as “effector memory cells”, have been suggested to be the cells that can provide this first line of defense against reinfection. Being able to elicit long-lived memory CD8⁺ T cell populations that are not only cytolytic, but multifunctional, in their ability to produce high levels of gamma interferon (IFN-γ) and tumor necrosis factor (TNF) may also be essential for protection. Therefore, molecules that induce high-frequency persisting multifunctional CD8 T cell populations that localize in mucosal tissues are likely a key factor in generating effective cellular immunity, and might offer considerable advantages in terms of protection if incorporated into a vaccine.

### BRIEF SUMMARY OF THE INVENTION

To enhance the cellular (Th1) immune response, aspects of the present invention utilize lipoteichoic acid (LTA) from beneficial intestinal bacteria, which activates antigen presenting cells by engaging Toll-like receptor (TLR)1/2 heterodimers. Additionally, an agonist antibody against OX40 (CD134) is administered to enhance memory T cells responses.

Aspects of the present invention provide a method for generating CD8+ T lymphocyte immune memory against one or more antigen. In various embodiments, a method for generating CD8+ T lymphocyte immune memory against one or more antigen is performed by administering to a subject an effective amount of LTA in combination with the one or more antigen. In some embodiments, one or more antibody against OX40 is also administered to aid in generation of CD8+ T lymphocyte immune memory. Aspects of the invention also provide for vaccines and formulations for vaccination comprising one or more antigen and LTA. In some embodiments, the vaccines and formulations further comprise at least one antibody against OX40. Additional aspects of the invention provide for methods of vaccination against a pathogen utilizing the disclosed vaccines and formulations.

### BRIEF DESCRIPTION OF THE DRAWINGS

FIG. 1 shows graphs of the stimulatory capacity of lipoteichoic acid (LTA) isolated from different lactobacilli. LTA isolated from different bacteria is used to stimulate RAW-GFP cells to evaluate the stimulatory capacity. The upper panel shows the expression of GFP by these cells upon stimulation with LTA (0.5 μg/ml), indicating an increase in NF-κB activity in these cells. The lower panel depicts the IL-12 production by these cells upon stimulation with LTA from different sources.

FIGS. 2A-2D show graphs of adjuvant capacity of LTA and anti-OX40 antibodies to mount a CD8+T cell response, as measured in spleen (A) and lungs (C) of mice 6 days post inoculation. Mice are injected intraperitoneally with ovalbumin (OVA) alone, OVA+LTA (L. acidophilus) alone, or OVA+LTA and anti-OX40 (OX86), which is given 1 day later. Expansion of OVA-specific CD8+ T cells is observed in spleen and lungs. OVA expressing Vaccinia Virus (VV-OVA) treated mice are used as a positive control and another non-LTA component (LTA-C) is used as a negative control for cell expansion. The ability of IFN-γ release is also evaluated in activated CD8+ T cells (CD44+CD8+) in spleen (B) and lungs (D) of mice 6 days post inoculation.

FIGS. 3A-3D show graphs of adjuvant capacity of LTA and anti-OX40 antibodies to mount a long-term CD8+ T cell response, as measured in spleen (A) and lungs (C) of mice 35 days post inoculation. Mice are injected intraperitoneally with ovalbumin (OVA) alone, OVA+LTA (L. acidophilus) alone, or OVA +LTA and anti-OX40 (OX86), which is given 1 day later as mentioned in FIG. 2. Presence of OVA-specific CD8+ T cells is observed in spleen and lungs at 35 days post inoculation. OVA expressing Vaccinia Virus (VV-OVA) treated mice are used as a positive control. The ability of IFN-γ release is also evaluated in activated CD8+ T cells (CD44+CD8+) in spleen (B) and lungs (D) of mice 35 days post inoculation.

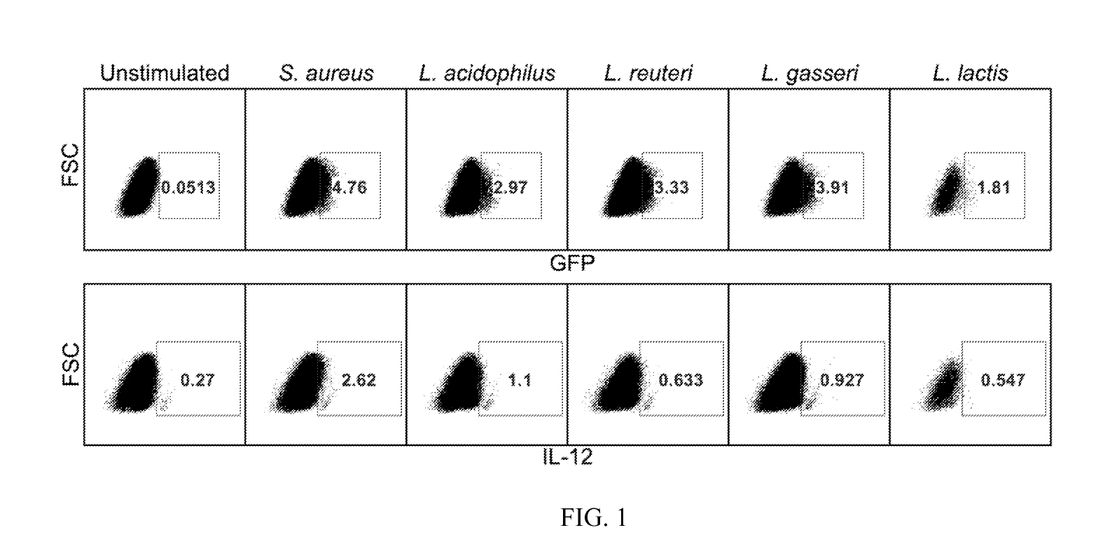

### DETAILED DISCLOSURE OF THE INVENTION

Before the present compositions and methods for vaccination are disclosed and described, it is to be understood that this invention is not limited to the particular process steps and materials disclosed herein as such process steps and materials may vary somewhat. It is also to be understood that the terminology employed herein is used for the purpose of describing particular embodiments only and is not intended to be limiting since the scope of the present invention will be limited only by the appended claims and equivalents thereof

It must also be noted that, as used in this specification and the appended claims, the singular forms “a”, “an” and “the” include plural referents unless the content clearly dictates otherwise. Additionally, the terms “comprising”, “consisting of” and “consisting essentially of” are defined according to their standard meaning. The terms may be substituted for one another throughout the instant application in order to attach the specific meaning associated with each term.

In describing and claiming the present invention, the following terminology will be used in accordance with the definitions set out below.

As used herein, the term “subject” refers to an animal. Typically, the terms “subject” and “patient” may be used interchangeably herein in reference to a subject. As such, a “subject” includes an animal that is being treated for a disease, being immunized, or the recipient of a mixture of components as described herein, such as a vaccine. The term “animal,” includes, but is not limited to, mouse, rat, dog, guinea pig, cow, horse, chicken, cat, rabbit, pig, monkey, chimpanzee, and human.

As used herein, the term “immune memory” or “memory” refers to the physiological condition characterized by long-lived antigen-specific lymphocytes with the ability to provide rapid recall responses upon future antigen experience. As would be understood by those skilled in the art, lymphocytes that provide such protection can be CD4+ or CD8+ T cells specific for the antigen.

As used herein, the term “antigen” refers to any molecule capable of generating an immune response, such as a peptide, polypeptide, protein, cell, cancer cell (such as a self-antigen associated with a cancer cell), live-attenuated pathogen, or heat-killed pathogen that has the potential to stimulate an immune response. Additionally, it is understood that “pathogen” refers to any organism capable of eliciting an immune response from a subject upon exposure or infection of the subject with the pathogen. It is contemplated that a given pathogen can be comprised of multiple antigens to which the subject's immune response may respond. It is also contemplated that a “pathogen” can refer to a cancer, virus, bacteria, or parasite, for example.

An antigen derived from a pathogen may be a subunit antigen, a peptide antigen, an inactivated pathogen, an attenuated pathogen or a recombinant antigen. “Virus” includes, for instance, hepatitis virus, RS virus, adenovirus, avulavirus, isavirus, canine distemper virus, influenza virus A-C, equine arteritis virus, Ebola virus, enterovirus, calicivirus, coronavirus, monkey immunodeficiency virus, thogotovirus, Deng virus, toga virus, avian infectious synovial bursa disease virus, avian pneumovirus (formerly turkey rhinotracheitis virus), nipah virus, Newcastle disease virus, pneumovirus, feline infectious peritonitis virus, feline leukemia virus, Norwalk virus, papilloma virus, papovavirus, parainfluenza virus types 1-3, parvovirus, picornavirus, human cytomegalovirus, human immunodeficiency virus, porcine respiratory and reproduction syndrome virus, flavivirus, henipavirus, hepadnavirus, herpes virus, Hendra virus, poliovirus, Marek's disease virus, metapneumovirus, morbillivirus, rhinovirus, rubulavirus, respirovirus, retrovirus, rotavirus, vaccinia virus, yellow fever virus, infectious rhinotracheitis virus, rinderpest virus, rabies virus, varicellovirus, encephalitis virus, rubella virus, measles virus and mumps virus. Influenza virus can be used as an antigen and an antigen derived therefrom can be HA, NA, M1, M2 and/or NP.

Bacterial antigens can be derived from, for instance, Actinobacillus pleuropneumoniae, Alloiococcus otitis, Influenza bacteria (including both those type-classifiable and those non-type-classifiable), Yersinia bacteria, Chlamydia psittaci, Campylobacter, Chlamydia pneumonia, Clostridia species, Vibrio cholerae, Salmonella choleraesuis, diphtheria bacteria, Pseudomonas species, Streptococcus gordonii, Streptococcus thermophilus, Streptococcus bovis, Streptococcus agalactiae, Chlamydia trachomatis, Mycobacterium avium group, Salmonella typhimurium, Pasteurella haemolytica, Pasteurella multocida, Mycobacterium tuberculosis, Streptococcus suis, Proteus vulgaris, Proteus mirabilis, Haemophilus somnus, Helicobacter pylori, Borrelia burgdorferi, Mycoplasma gallisepticum, Moraxella catarrhalis, Leptospira interrogans, Staphylococcus aureus, Streptococcus pyogenes, Neisseria meningitidis, Shigella, Streptococcus equi, Escherichia coli, anthrax, typhoid bacteria, Clostridium tetani, Streptococcus pneumoniae, Bordetella pertussis, Staphylococcus epidermidis, Streptococcus faecalis, Streptococcus viridans, and Neisseria gonorrhoeae.

Parasitic antigens can be derived from, for example, Entamoeba histolytica, Plasmodium, Leishmania major, Ascaris, Trichuris, Giardia, Schistosoma, Cryptosporidium, Trichomonas, Toxoplasma, and Pneumocystis carinii.

As used herein, the term “vaccine” or “immunizing formulation” refers to any composition comprising a fragment of one or more antigens or whole antigens wherein the composition stimulates an immune response to the antigen or antigens. Thus, a vaccine refers to any composition that is administered to a subject with the goal of establishing an immune response and/or immune memory to a particular pathogen. It is also contemplated that the vaccine compositions can comprise other substances designed to increase the ability of the vaccine to generate an immune response. For example, a typical vaccine can comprise an antigen plus an adjuvant, such as, but not limited to, LTA and anti-OX40 antibody. It is also contemplated that the vaccines disclosed herein can be therapeutic or prophylactic. Thus, for example, the vaccines disclosed herein can be used to prevent an infection such as, but not limited to, viral infection. Alternatively, the vaccines disclosed herein can be used therapeutically to treat an individual with cancer or a chronic infection such as, but not limited to, HIV. It is also contemplated that the present invention can provide more than one antigen in the mixtures of compositions herein disclosed. For example, a mixture can comprise a peptide of a protein of a pathogen and a second peptide of the same related pathogen. Also, the disclosed methods can comprise the simultaneous or separate administration of multiple vaccines. Thus, the present invention further includes the administration of a second, third, fourth, etc. antigen, wherein the second, third, fourth, etc. antigen is administered in a separate vaccine for administration at the same time as or 1, 2, 3, 4, 5, 6, 10, 14, 18, 21, 30, 60, 90, 120, 180 or 360 days (or any number of days in between) after the first antigen.

Additionally, the antigens provided in the mixture for vaccines or immunization protocols can come from the same, different or unrelated pathogens. Thus, the antigens may be the same antigen, or the antigens may be related to heterologous antigens. For example, the present invention provides methods of producing memory T lymphocytes or protection comprising administering to a subject a mixture comprising a first antigen related to a first pathogen and a second antigen related to a second pathogen, in addition to LTA and one or more anti-OX40 antibody.

The term “effective amount,” as applied to the compositions described herein, means the quantity necessary to render the desired therapeutic or immunological result. For example, an effective amount of an antigen administered in a vaccine is a dosage level effective to initiate, expand and maintain a memory T lymphocyte level that provides an effective or efficient immune response when challenged by the same antigen at a later time.

Some variation in dosage will necessarily occur depending upon many factors that would be known by those skilled in the art, and the physician or other individual administering such a vaccine or immunization will, in any event, determine the appropriate dosage for an individual patient. The term “pharmaceutically acceptable,” as used herein with regard to compositions and formulations, means approved by a regulatory agency of the Federal or a state government or listed in the U.S. Pharmacopeia or other generally recognized pharmacopeia for use in animals and/or in humans.

The term “carrier” refers to a diluent, excipient, and/or vehicle with which the antigen(s), LTA, and anti-OX40 antibodies are administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include, but are not limited to, starch, glucose, sucrose, gelatin, lactose, malt, rice, flour, chalk, silica gel, sodium stearate, glycerol monostearate, talc, sodium chloride, glycerol, propylene, glycol, water, ethanol and the like. The compositions and formulations described herein may also contain wetting or emulsifying agents, suspending/diluting agents, pH buffering agents, or agents for modifying or maintaining the rate of release of the antigen and/or LTA and/or anti-OX40 antibody. These compositions/formulations and vaccines can take the form of solutions, suspensions, emulsions, tablets, pills, capsules, powders, sustained-release formulations and the like. Formulations can include standard carriers such as pharmaceutical grades of mannitol, lactose, sodium saccharine, starch, magnesium stearate, cellulose, magnesium carbonate, etc. Such compositions and vaccines will contain an effective amount of the antigen(s) and LTA and/or antibody together with a suitable amount of carrier so as to provide the proper form to the patient based on the mode of administration to be used.

If for intravenous administration, the vaccines, compositions and formulations can be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachette indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

Aspects of the present invention provide a method for generating CD8+ T lymphocyte immune memory against one or more antigen. In various embodiments, a method for generating CD8+ T lymphocyte immune memory against one or more antigen is performed by administering to a subject an effective amount of LTA in combination with the one or more antigen. In various embodiments, the one or more antigen is a recombinant or synthetic antigen derived from a pathogen.

In another aspect, the present invention provides methods for vaccinating a subject against a pathogen. The methods comprise administering to the subject a composition comprising LTA and one or more recombinant or synthetic antigen derived from the pathogen. In some embodiments, the subject is a human.

In embodiments of the aspects provided, LTA is isolated from Lactobacillus acidophilus, Lactobacillus reuteri, Lactobacillus gasseri, and/or Lactobacillus lactis. Also, the method aspects provided by the present invention can further include administering an effective amount of OX40 antibody to the subject. The OX40 antibody can be administered simultaneously, sequentially or in a combined composition with LTA and the one or more antigen. OX40 (also referred to as CD134, TNFRSF4 and ACT35) is a 50 kilodalton (KDa) glycoprotein and a member of the tumor necrosis factor receptor superfamily (TNFRSF) that is expressed on immune cells, particularly CD4⁺ and CD8⁺ T cells. The ligand for OX40, OX40L (also referred to as TXGP1L, TNFSF4, CD252), has been reported to be expressed on endothelial cells, activated antigen presenting cells including macrophages, dendritic cells, B cells, and natural killer cells. Engagement of CD40 on antigen presenting cells increases OX40L expression, as can lipopolysaccharide (LPS). Expression of OX40 on T cells can be induced following signaling via the T cell antigen receptor. For example, OX40 is expressed on recently activated T cells at the site of inflammation. Thus, CD4⁺ and CD8⁺T cells can up-regulate OX40 under inflammatory conditions. OX40 can promote a number of activities in T cells including causing their division, survival, and promoting their effector function (e.g., to kill virally infected cells). Agonist reagents (antibodies, fusion proteins, and other modalities that cross-link OX40 and promote intracellular signaling) can be used to stimulate OX40 and enhance T cell immunity. As such, aspects of the present invention also contemplate use of any type of agonist reagent to OX40, such as fusion proteins and cross-linking agents.

In additional aspects, the present invention provides formulations for vaccination and vaccines comprising one or more antigen and LTA. In embodiments of this aspect of the invention, the formulations/vaccines can further include at least one pharmaceutically acceptable carrier. In some embodiments, the formulations/vaccines further comprise one or more anti-OX40 antibody. Embodiments of this aspect of the invention further provide for methods of vaccinating a subject, such as a human, by administering a therapeutically effective amount of the formulation.

Thus, the following non-limiting embodiments are provided:

1. A method for generating CD8+ T lymphocyte immune memory against one or more antigen, the method comprising administering to a subject an effective amount of lipoteichoic acid in combination with the one or more antigen.

2. The method according to embodiment 1, wherein the antigen is a recombinant or synthetic antigen derived from a pathogen.

3. The method according to any one of embodiments 1-2, wherein the lipoteichoic acid is isolated from Lactobacillus acidophilus.

4. The method according to any one of embodiments 1-2, wherein the lipoteichoic acid is isolated from Lactobacillus reuteri.

5. The method according to any one of embodiments 1-2, wherein the lipoteichoic acid is isolated from Lactobacillus gasseri.

6. The method according to any one of embodiments 1-2, wherein the lipoteichoic acid is isolated from Lactobacillus lactis.

7. The method according to any one of embodiments 1-6, further comprising administering to the subject an effective amount of one or more anti-OX40 antibody.

8. The method according to embodiment 7, wherein the anti-OX40 antibody is administered simultaneously with lipoteichoic acid and the antigen.

9. The method according to any one of embodiments 1-8, wherein the subject is a human.

10. A method for vaccinating a subject against a pathogen, comprising administering to the subject a composition comprising lipoteichoic acid and a recombinant or synthetic antigen derived from the pathogen.

11. The method of embodiment 10, wherein the composition further comprises one or more anti-OX40 antibody.

12. The method according to any one of embodiments 10-11, wherein the subject is a human.

13. A formulation for vaccination, comprising an antigen and lipoteichoic acid.

14. The formulation of embodiment 13, wherein the antigen is a recombinant or synthetic antigen.

15. The formulation according to any one of embodiments 13-14, further comprising one or more anti-OX40 antibody.

16. The formulation according to any one of embodiments 13-15, further comprising at least one pharmaceutically acceptable carrier.

17. A method of immunizing a subject, comprising administering to the subject a therapeutically effective amount of the formulation according to any one of embodiments 13-16.

18. The method of embodiment 17, wherein the subject is a human.

19. A vaccine, comprising a recombinant or synthetic antigen, lipoteichoic acid, and one or more anti-OX40 antibody.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Isolation of Lipoteichoic Acid (LTA) from Lactobacilli

To test the immune-stimulatory capacity of LTA isolated from different lactobacilli, LTA is isolated using a butanol extraction. In brief, bacteria grown in appropriate media, such as MRS (L. acidophilus, L. gasseri, L. reuteri) or M17 (Lactococcus lactis), is washed twice with phosphate buffered saline (PBS) to remove any traces of media and then frozen at −80° C. overnight. The defrosted bacteria are sonicated (Power 50%, 10 impulses of 30 seconds) to disrupt the cell wall in citrate buffer (pH 4). The sonicated material is then mixed with an equal volume of n-butanol on vortex for 1 hour. Centrifugation of bacterial material results in a biphasic separation, and lyophilization of the upper layer yields LTA as powder. The powder is then dissolved in PBS. A genetically engineered murine macrophage cell line (Raw 264.7-GFP) that expresses green fluorescent protein (GFP) under a NF-κB driven promoter is used to compare the stimulatory capacity of isolated LTA and LTA isolated from Staphylococcus aureus as a control. Upon treatment, an equivalent capacity of isolated LTA compared to the commercial LTA is observed.

### EXAMPLE 2

### LTA and Anti-OX40 Treatment Enhances the Frequency of Effector CD8 T Cells in the Lungs and Spleen after Protein Vaccination

To assess the potential of targeting LTA to promote protective resident virus-specific memory CD8⁺ T cell populations, systemic priming is focused on so as not to bias the generation of mucosal associated T cells. Additionally, many vectors that are being used as vaccine vehicles are being tested systemically because of possible safety concerns regarding mucosal immunization, as well as simple logistical issues associated with the efficiency of mucosal versus systemic vaccination.

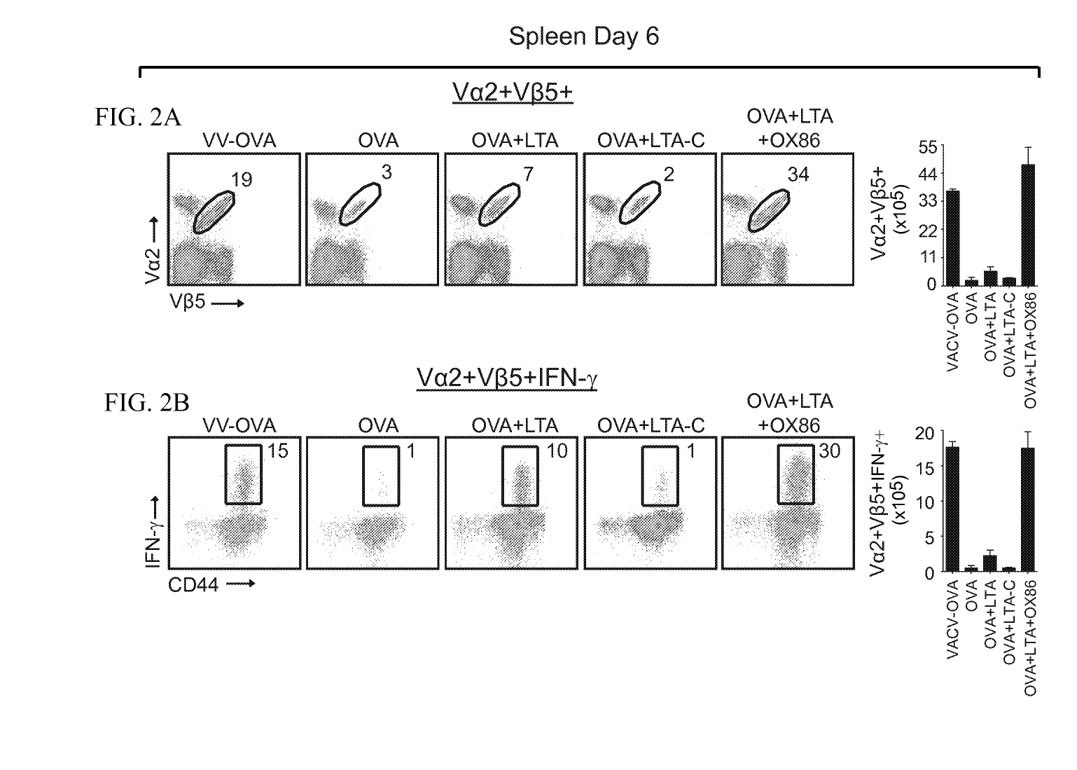

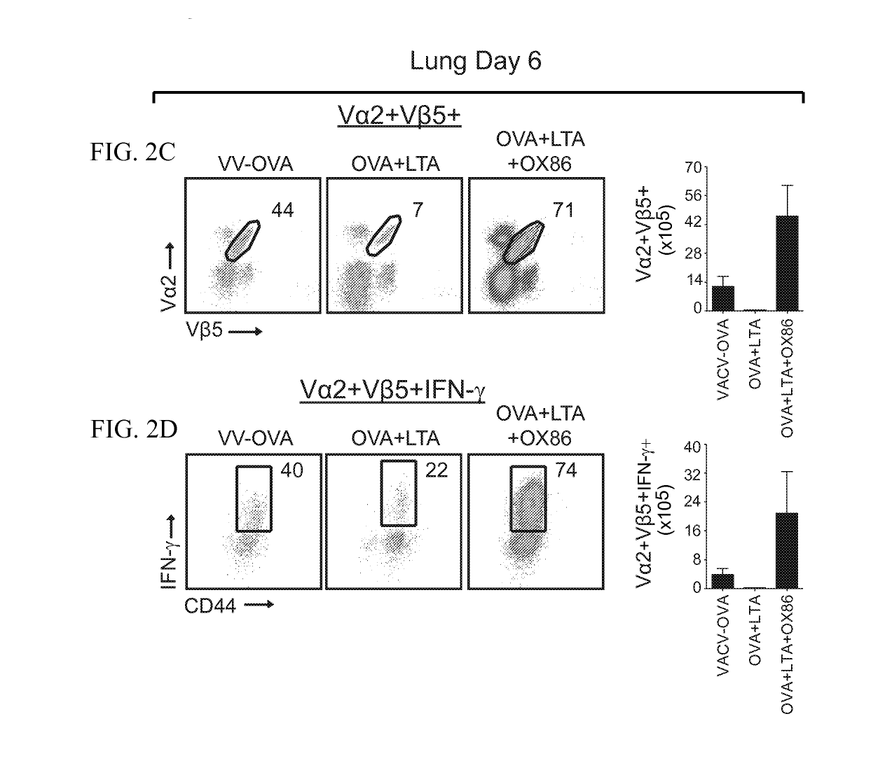

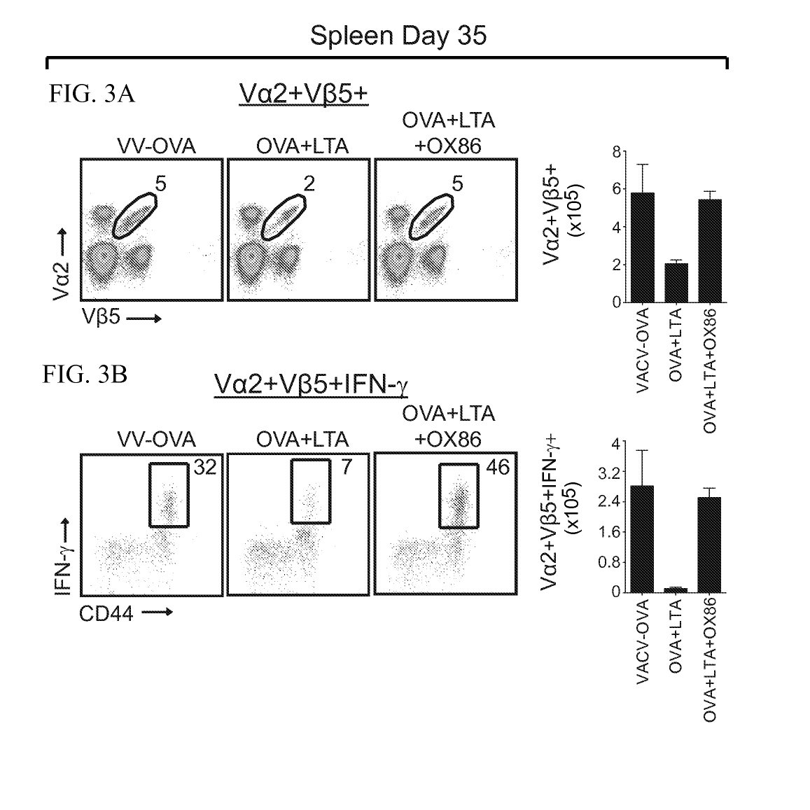

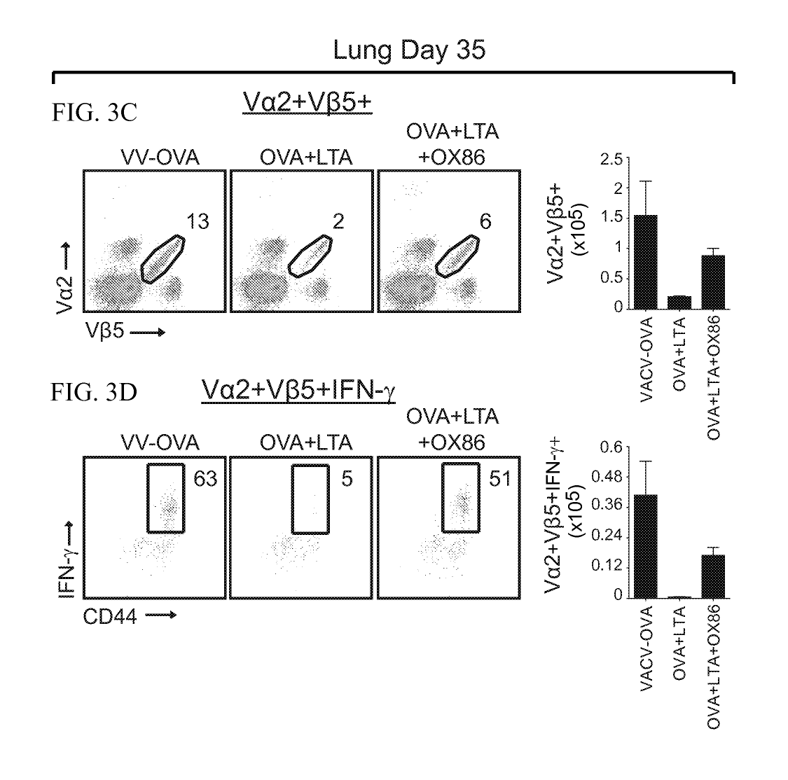

Mice are injected intraperitoneally with ovalbumin (OVA) alone, OVA+LTA (L. acidophilus) alone, or OVA+LTA and anti-OX40, which is given 1 day later. Immunologic memory is the most important feature of any vaccination protocol, but before focusing on this aspect of the response, it is determined if targeting LTA and OX40 with protein immunization replicates the systemic and peripheral effector responses observed with live virus immunization. LTA treatment alone strongly boosts the number of OVA-specific primary effector CD8⁺ T cells and, in particular, the multifunctional subset that produces high levels of TNF and IFN-γ; this is observed not only in the spleen, but also significantly in the lungs (FIGS. 2 and 3). Most importantly, a strong accumulation of OVA-reactive effector CD8⁺ T cells is observed when LTA is combined with anti-OX40 treatment. This suggests that LTA and OX40 signals promote mucosal immunologic memory.

The goal of vaccination is the generation of a strong immune response to the administered antigen that is able to provide long-term protection against infection. To achieve this goal, antigen is often mixed with adjuvants, especially if the antigen is a purified protein or other less immunogenic fraction of a pathogen. Alum, a commonly used adjuvant, only elicits a strong Th2 response to augment a protective humoral response, which is insufficient to protect against intracellular pathogens. However, for the development of vaccines against intracellular pathogens, a strong Th1 response is needed. Additionally, like the humoral response (antibody production), the presence of effector memory cells at mucosal sites is needed to reduce the time between infection and pathogen clearance. Engagement of OX40 on T cells has been shown to result in an increase in effector T cells. As such, the present invention utilizes LTA and anti-OX40 antibodies as adjuvants to generate a strong, long-term immune response to antigens.

By combining LTA and an agonist OX40 antibody for vaccination, the present invention has enhanced the quality of immunization against peptide antigens, more so than a live vaccine, which is considered to be a benchmark for immune protection. In agreement with the purposes of the invention, the present invention provides a combination of adjuvants to enhance the existing vaccine by changing its composition, which enhances the response to a subunit vaccine comparable to a live attenuated vaccine. LTA provides the required stimulus to antigen presenting cells, and simultaneous engagement of OX40 enhances the vaccine response considerably.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1-19. (canceled)

20. A method for generating CD8+ T lymphocyte immune memory against one or more antigen, the method comprising administering to a subject an effective amount of lipoteichoic acid in combination with the one or more antigen.

21. The method of claim 20, wherein the antigen is a recombinant or synthetic antigen derived from a pathogen.

22. The method of claim 20, wherein the lipoteichoic acid is isolated from Lactobacillus acidophilus, Lactobacillus reuteri, Lactobacillus gasseri or Lactobacillus lactis.

23. The method of claim 20, further comprising administering to the subject an effective amount of one or more anti-OX40 antibody.

24. The method of claim 23, wherein the anti-OX40 antibody is administered simultaneously with lipoteichoic acid and the antigen.

25. The method of claim 22, further comprising administering to the subject an effective amount of one or more anti-OX40 antibody.

26. The method of claim 25, wherein the anti-OX40 antibody is administered simultaneously with lipoteichoic acid and the antigen.

27. The method of claim 25, wherein the subject is a human. cm 28. A method for vaccinating a subject against a pathogen, comprising administering to the subject a composition comprising lipoteichoic acid and a recombinant or synthetic antigen derived from the pathogen.

29. The method of claim 28, wherein the composition further comprises one or more anti-OX40 antibody.

30. The method of claim 29, wherein the subject is a human.

31. A formulation for vaccination, comprising an antigen and lipoteichoic acid.

32. The formulation of claim 31, wherein the antigen is a recombinant or synthetic antigen.

33. The formulation of claim 31, further comprising one or more anti-OX40 antibody.

34. The formulation of claim 31, further comprising at least one pharmaceutically acceptable carrier.

35. The formulation of claim 33, Further comprising at least one pharmaceutically acceptable carrier.

36. A method of immunizing a subject, comprising administering to the subject a therapeutically effective amount of the formulation of claim 31.

37. The method of claim 36, wherein the subject is a human.

38. A vaccine comprising a recombinant or synthetic antigen, lipoteichoic acid, and one or more anti-OX40 antibody.
